(19) United States
(12) Patent Application Publication
(10) Pub. No.: US 2026/0137361 A1
(43) Pub. Date: May 21, 2026

(54) X-RAY FLUOROSCOPIC IMAGING APPARATUS

(71) Applicant: SHIMADZU CORPORATION, Kyoto-shi (JP)

(72) Inventors: Kazuyuki MATSUDA, Kyoto-shi (JP); Yumi TSUTSUMIDA, Kyoto-shi (JP)

(21) Appl. No.: 19/107,815

(22) PCT Filed: May 1, 2023
(86) PCT No.: PCT/JP2023/017041

§ 371 Date: Feb. 11, 2026

(30) Foreign Application Priority Data
Aug. 30, 2022 (JP) 2022-136826

(51) Int. Cl. A61B 6/00; A61B 6/42; A61B 6/46; A61B 6/50

(52) U.S. Cl. CPC A61B 6/487; A61B 6/42; A61B 6/4441; A61B 6/463; A61B 6/504; A61B 6/54

(57) ABSTRACT

An X-ray fluoroscopic imaging apparatus includes: a C-arm 17 that is rotatable, and supports an X-ray tube 13 and an X-ray detector 15 such that the X-ray tube 13 and the X-ray detector 15 are opposite to each other; an image generator 43 that generates X-ray images L using detection signals of the X-ray detector 15; and an image display device 11 that displays the X-ray images L. The image display device 11 includes: an image display unit 49 that can display information display images D in a row and a column in a matrix; an input unit 51 that inputs an instruction to change a position at which image information K is displayed in each of the information display images D such that at least two of the X-ray images L are displayed adjacently in the respective information display images D; and an image display control unit 57 that causes the image display unit 49 to display the X-ray images L adjacently based on a content of the instruction.

### TECHNICAL FIELD

The present invention relates to an X-ray fluoroscopic imaging apparatus.

### BACKGROUND ART

In a medical field, for example, an X-ray fluoroscopic imaging apparatus that performs X-ray fluoroscopy A X-ray imaging has become indispensable for performing an angiographic examination by manipulating a catheter. In this operative procedure, X-rays are emitted to a cardiovascular region of a subject from a certain direction for fluoroscopic imaging. An operator operates a catheter appropriately while referring to X-ray image data obtained by the fluoroscopic imaging and continues a medical operation.

Conventional X-ray fluoroscopic imaging apparatuses include a table on which a subject is placed, an image pickup system that includes an X-ray tube and an X-ray detector, a C-shaped arm (C-arm) that supports the image pickup system, and the like. The X-ray tube and the X-ray detector are provided on one end and another end of the C-arm. The C-arm is disposed such that the X-ray tube and the X-ray detector are arranged oppositely with the subject interposed therebetween. The C-arm can slide and move along an arc path following a shape of the arm. The C-arm is supported by a rotatable rotation mechanism, and rotatable around a predetermined axis according to a rotation of the rotation mechanism. Thus, a user can use the C arm to emit X-rays from a desired angle, and obtain a radiogram through fluoroscopy or imaging at that angle.

As an apparatus that obtains radiograms of a subject in real time from two different directions, an X-ray fluoroscopic imaging apparatus (biplane X-ray fluoroscopic imaging apparatus) including two C-arms that support an image pickup system has been proposed particularly in recent years (Patent Literature 1, for example). Use of the biplane X-ray fluoroscopic imaging apparatus enables fluoroscopic imaging to be performed on a predetermined area of interest simultaneously from two directions with a single injection of a contrast agent. Such an X-ray fluoroscopic imaging apparatus is also called a cardiovascular systems (CVS) device, and is applied to operative procedures using a catheter for a cardiovascular system.

When performing angiographic imaging with the biplane X-ray fluoroscopic imaging apparatus, a user inserts a catheter into a subject and allows the catheter to reach an area of interest. Then, the user injects a contrast agent from the catheter into a blood vessel in the area of interest and performs X-ray imaging with a contour of the blood vessel visualized, and obtains X-ray image data of a contrast image (reference image). The X-ray image data of the reference image is displayed on an image display device, and at the same time, X-ray image data (acquired image) obtained in real time is displayed on the image display device. The user operates the catheter and continues the medical operation while visually comparing the reference image with the contrast agent visualized and the acquired image showing a most recent area of interest.

### CITATION LIST

### Patent Literature

Patent Literature 1: JP 2016-158658 A

### SUMMARY OF INVENTION

### Technical Problem

However, the conventional example having such a configuration has the problem as described below.

The X-ray image data obtained by the X-ray fluoroscopic imaging apparatus is displayed on the image display device, with an information display region attached to an image display region being a region in which the X-ray image is displayed. The information display region, which is a region in which information on an X-ray image, for example, an X-ray emission condition or an X-ray emission angle, is displayed, is indispensable in the X-ray image data. A positional relation between the image display region and the information display region in each piece of the X-ray image data is fixed. As an example, the information display region is arranged on the left of the image display region. When the X-ray image data of the acquired image and the X-ray image data of the reference image are displayed in a row on the image display device, the information display region of the acquired image or the information display region of the reference image is displayed between the image display region of the acquired image and the image display region of the reference image.

With this display mode, the image display region inevitably comes into a user's field of vision every time the user visually compares the X-ray image of the acquired image and the X-ray image of the reference image. As a result, the information displayed in the information display region becomes an obstacle to comparison and examination of the X-ray images for the user, which hinders a quick medical operation and increases a burden imposed on the user.

The present invention has been made in view of such circumstances, and an object thereof is to provide an X-ray fluoroscopic imaging apparatus that enables easy comparison and examination of a plurality of displayed X-ray images.

### Solution to Problem

In order to achieve such an object, the present invention adopts the following configuration.

A first aspect of the present invention relates to an X-ray fluoroscopic imaging apparatus including: an X-ray tube that emits an X-ray to a subject; an X-ray detector that detects an X-ray transmitted through the subject; an arm that supports the X-ray tube and the X-ray detector such that the X-ray tube and the X-ray detector are opposite to each other; an arm rotation mechanism that rotates the arm around a predetermined axis; an X-ray image generator that generates an X-ray image using a detection signal output by the X-ray detector; and an X-ray image display device that displays the X-ray image. The X-ray image display device includes: an image display unit that is capable of displaying, in a row and a column in a matrix, information display images each containing an X-ray image display region in which the X-ray image showing a predetermined area of interest is displayed and an image information display region in which information on the X-ray image is displayed; an information display region change instruction unit that inputs an instruction to change a position of the image information display region in each of the information display images displayed in the row and the column on the image display unit, such that at least two of the X-ray image display regions are displayed adjacently; and an image display control unit that controls the image display unit such that based on a content of the instruction input to the information display region change instruction unit, the position of the image information display region in the information display image is changed to display the X-ray image display regions adjacently.

A second aspect of the present invention relates to an X-ray fluoroscopic imaging apparatus including: an X-ray tube that emits an X-ray to a subject; an X-ray detector that detects an X-ray transmitted through the subject; an arm that supports the X-ray tube and the X-ray detector such that the X-ray tube and the X-ray detector are opposite to each other; an arm rotation mechanism that rotates the arm around a predetermined axis; an X-ray image generator that generates an X-ray image using a detection signal output by the X-ray detector; and an X-ray image display device that displays the X-ray image. The X-ray image display device includes: an image display unit that is capable of displaying, in a row and a column in a matrix, information display images each containing an X-ray image display region in which the X-ray image showing a predetermined area of interest is displayed and an image information display region in which information on the X-ray image is displayed; an information display position setting unit that sets a position of the image information display region in the information display image in advance so as to display the X-ray image display regions adjacently according to a pattern with a positional relation in which the information display images are displayed in the row and the column; and an image display control unit that controls the image display unit such that after the pattern with the positional relation in which the information display images are displayed in the row and the column is determined, the position of the image information display region in the information display image corresponds to a position set by the information display position setting unit.

### Advantageous Effects of Invention

According to the first aspect of the present invention, the X-ray fluoroscopic imaging apparatus includes an image display device 11 that displays the X-ray images showing the predetermined area of interest, and the X-ray image display device includes the image display unit, an input unit 51, and the image display control unit. The information display images each containing the X-ray image display region and the image information display region are displayed in the row and the column in the matrix on the image display unit of the X-ray image display device.

The input unit 51 is capable of inputting the instruction to change a position of the X-ray image display region and the position of the image information display region in the information display image such that at least two of the X-ray image display regions are displayed adjacently. The image display control unit causes the image display unit to display the X-ray image display regions adjacently according to the instruction input to the input unit 51.

That is, unlike conventional apparatuses with an unchanged positional relation between the X-ray image display region and image information K in the information display image, the X-ray fluoroscopic imaging apparatus according to the first aspect is capable of changing the positional relation between the X-ray image display region and the image information display region in the information display image. Even when a pattern in which the plurality of information display images are arranged in the matrix on the image display unit is changed as needed according to a request of a user, a setting to change anew a positional relation between an X-ray image L and the image information K in each of the information display images can be made such that the X-ray image display regions are adjacent to each other in the changed arrangement pattern of the information display images. Accordingly, when the X-ray images in the plurality of information display images are visually compared with each other, the arrangement pattern of the information display images is changed according to the request of the user, and at the same time, presence of the image information display region between the X-ray images to be compared and examined can be reliably avoided. Thus, it is possible to avoid the image information display region becoming an obstacle to a motion in visually comparing the X-ray images; therefore, it is possible to perform a motion in comparing and examining the X-ray images more easily.

According to the second aspect of the present invention, the X-ray fluoroscopic imaging apparatus includes the X-ray image display device that displays the X-ray images showing the predetermined area of interest, and the X-ray image display device includes the image display unit, the information display position setting unit, and the image display control unit.

The information display images each containing the X-ray image display region and the image information display region are displayed in the row and the column in the matrix on the image display unit.

The information display position setting unit sets the position of the image information display region in the information display image in advance so as to display the X-ray image display regions adjacently according to the pattern with the positional relation in which the information display images are displayed in the row and the column. The image display control unit causes the image display unit to display the X-ray image display regions adjacently based on the position, set by the information display position setting unit, of the image information display region in the information display image.

As described above, in the X-ray fluoroscopic imaging apparatus according to Item 2, the position of the image information display region in the information display image can be set in advance before the X-ray image is generated. That is, since the X-ray image display regions can be displayed adjacently without a motion in changing the position of the image information display region during an ongoing medical operation, it is possible to perform the motion in comparing and examining the X-ray images more easily, and also to reduce a procedure and time required for the medical operation.

### BRIEF DESCRIPTION OF DRAWINGS

FIG. 1 is a front view illustrating an overall configuration of an X-ray fluoroscopic imaging apparatus according to Example 1.

FIG. 2 is a right-side view illustrating the overall configuration of the X-ray fluoroscopic imaging apparatus according to Example 1.

FIG. 3 is a functional block diagram illustrating a schematic configuration of the X-ray fluoroscopic imaging apparatus according to Example 1.

FIG. 4 is a diagram illustrating a configuration of an information display image according to Example 1.

FIG. 5 is a diagram illustrating an example of a pattern in which a plurality of information display images are arranged, according to Example 1.

FIG. 6 is a flowchart illustrating a functional operation of the X-ray fluoroscopic imaging apparatus according to Example 1.

FIG. 7 is a diagram illustrating step S1 according to Example 1.

FIG. 8 is a diagram illustrating step S2 according to Example 1.

FIG. 9 is a diagram illustrating step S3 according to Example 1.

FIG. 10 is a diagram illustrating step S4 according to Example 1.

FIG. 11 is a diagram illustrating step S4 according to Example 1.

FIG. 12 is a flowchart illustrating a functional operation of an X-ray fluoroscopic imaging apparatus according to Example 2.

FIG. 13 is a diagram illustrating step FO according to Example 2.

FIG. 14 is a diagram illustrating step FO according to Example 2.

FIG. 15 is a diagram illustrating step FO according to Example 2.

FIG. 16 is a diagram illustrating step F2 according to Example 2.

FIG. 17 is a diagram illustrating step F3 according to Example 2.

FIG. 18 is a diagram illustrating a problem of a conventional configuration.

FIG. 19 is a diagram illustrating a configuration of an information display image according to a comparative example. FIG. 19(a) is a diagram illustrating a configuration of an information display image including an acquired image, and FIG. 19(b) is a diagram illustrating a configuration of an information display image including a reference image.

FIG. 20 is a diagram illustrating a problem of the Comparative example.

FIG. 21 is a diagram illustrating a problem of the comparative example.

FIG. 22 is a diagram illustrating a layout of information display images in an initial state according to a modified example.

FIG. 23 is a diagram illustrating a state in which the layout of the information display images according to the modified example is changed.

### DESCRIPTION OF EMBODIMENTS

### Example 1

Hereinafter, Example 1 of the present invention will be described with reference to the drawings.

### <Description of Overall Configuration>

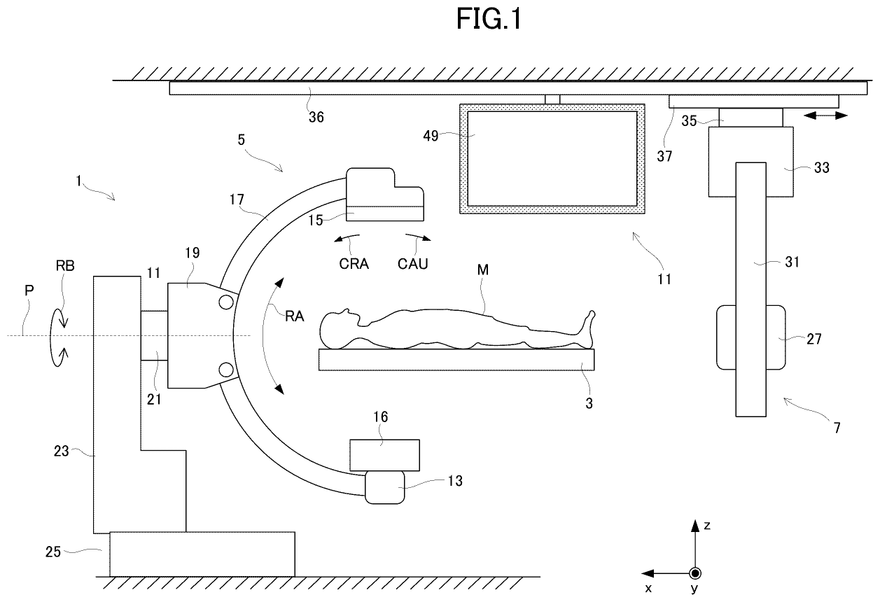

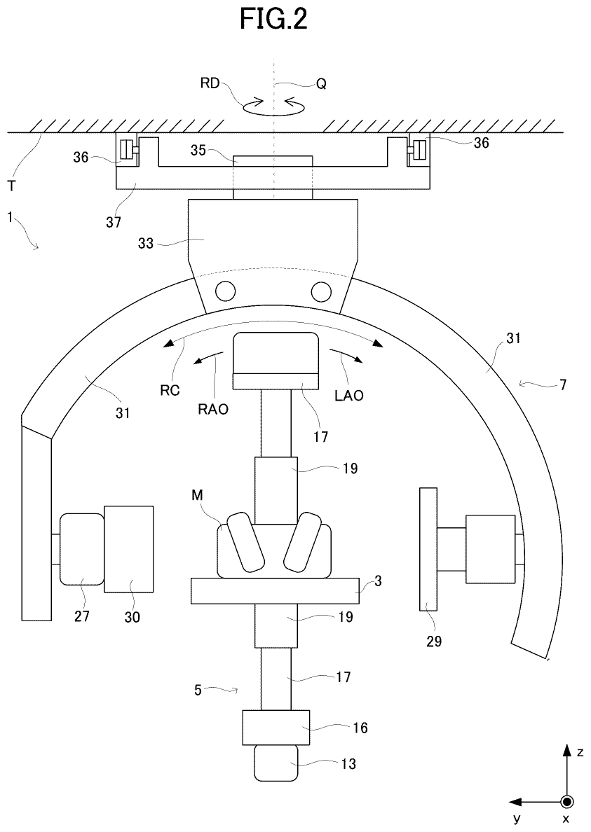

As illustrated in FIGS. 1 and 2, an X-ray fluoroscopic imaging apparatus 1 according to Example 1 includes a table 3, a first image pickup mechanism 5, and a second image pickup mechanism 7. A subject M in a supine posture is placed on the table 3. FIG. 1 illustrates a state in which soles of the subject M face this side.

With the first image pickup mechanism 5, X-rays are emitted to the subject M perpendicularly, for example. The first image pickup mechanism 5 includes an X-ray tube 13, an X-ray detector 15, a C-arm 17, a slide mechanism 19, and a rotation mechanism 21.

The X-ray tube 13 emits X-rays to the subject M. The X-ray detector 15 detects the X-rays emitted from the X-ray tube 13 and obtains an electric signal through light conversion. The X-ray tube 13 and the X-ray detector 15 are arranged oppositely with the table 3 interposed therebetween. The X-ray tube 13 and the X-ray detector 15 constitute an image pickup system. A collimator 16, which is disposed on the X-ray tube 13, restricts the X-ray emitted from the X-ray tube 13 to a predetermined shape. Examples of the shape of the X-ray restricted by the collimator 16 include a cone shape to be formed into a pyramid shape.

The C-arm 17 has a substantially C-shape, which is curved. The X-ray tube 13 is provided on one end of the C-arm 17, and the X-ray detector 15 is provided on another end of the C-arm 17.

The slide mechanism 19 slidably supports the C-arm 17. That is, the C-arm 17 slides and moves along an arc path for the C-arm 17 indicated by reference sign RA. The arc path RA is a path around an axis in a y direction (short direction of the table 3), which conforms to the arm shape of the C-arm 17.

The rotation mechanism 21, which is disposed on a side surface of a columnar support 23, rotatably supports the slide mechanism 19. The rotation mechanism 21 is rotatable around a horizontal axis P (hereinafter, also referred to as “around an axis of a body”) parallel with an x direction (long direction of the table 3). The C-arm 17 held by the rotation mechanism 21 via the slide mechanism 19 rotates around the axis in the x direction according to a rotation of the rotation mechanism 21. An arc path around the horizontal axis P is indicated by reference sign RB.

The C-arm 17 is rotatable around two orthogonal axes along the arc path RA and the arc path RB, so that X-rays can be emitted onto the subject M from any directions. FIG. 1 illustrates a state in which the X-ray tube 13 emits X-rays to the subject M in a z direction (direction orthogonal to a placement surface of the table 3).

The columnar support 23 is supported by a pedestal 25 disposed on a floor, and is horizontally movable in the y direction (short direction of the table 3). The rotation mechanism 21 supported by the columnar support 23, the slide mechanism 19, and the C-arm 17 move in the y direction according to a horizontal movement of the columnar support 13.

With the second image pickup mechanism 7, X-rays are emitted to the subject M from a direction different from that of the first image pickup mechanism 5. With the second image pickup mechanism 7, X-rays are emitted horizontally, for example. The second image pickup mechanism 7 includes an X-ray tube 27, an X-ray detector 29, a C-arm 31, a slide mechanism 33, and a rotation mechanism 35.

The X-ray tube 27 emits X-rays to the subject M. The X-ray detector 29 detects the X-rays emitted from the X-ray tube 27 and obtains an electric signal through light conversion. The X-ray tube 27 and the X-ray detector 29 are arranged oppositely with the table 3 interposed therebetween, and constitute an image pickup system. A collimator 30, which is disposed on the X-ray tube 27, restricts an X-ray emitted from the X-ray tube 27 to a predetermined shape. The C-arm 31 has a substantially C-shape, which is curved. The X-ray tube 27 is provided on one end of the C-arm 31, and the X-ray detector 29 is provided on another end of the C-arm 31. The slide mechanism 33 slidably supports the C-arm 31. That is, the C-arm 31 slides and moves along an arc path for the C-arm 31 indicated by reference sign RC.

The rotation mechanism 35, which is disposed on a top surface of the slide mechanism 33, rotatably holds up the slide mechanism 33. The rotation mechanism 35 is rotatable around a vertical axis Q parallel with the z direction (direction orthogonal to the placement surface of the table 3). The C-arm 31 held by the rotation mechanism 35 via the slide mechanism 33 rotates around the axis in the z direction according to a rotation of the rotation mechanism 35. An arc path around the vertical axis Q is indicated by reference sign RD. The C-arm 31 is rotatable around two orthogonal axes along the arc path RC and the arc path RD, so that X-rays can be emitted onto the subject M from any directions.

The rotation mechanism 35 is suspended from a ceiling T via a rail 36 and an overhead traveling unit 37. The rail 36 extends along the ceiling T in the x direction. The overhead traveling unit 37 is connected to the rotation mechanism 35. The overhead traveling unit 37 can reciprocate in the x direction along the rail 36.

As described above, the X-ray fluoroscopic imaging apparatus 1 according to Example 1 is what is called a biplane X-ray fluoroscopic imaging apparatus including the first image pickup mechanism 5 and the second image pickup mechanism 7. By using the first image pickup mechanism 5 and the second image pickup mechanism 7, X-rays can be emitted onto the subject M simultaneously from different angles.

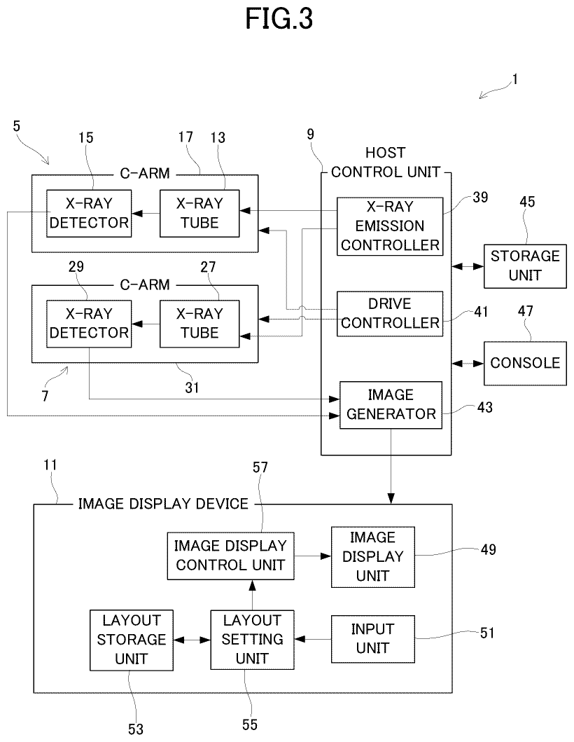

As illustrated in FIG. 3, the X-ray fluoroscopic imaging apparatus 1 further includes a host control unit 9 and an image display device 11. The host control unit 9 includes, for example, an information processing unit such as a central processing unit (CPU), and integrally controls various components in the X-ray fluoroscopic imaging apparatus 1. The host control unit 9 includes an X-ray emission controller 39, a drive controller 41, and an image generator 43.

The X-ray emission controller 39 individually controls each of the X-ray tube 13 and the X-ray tube 27. The X-ray emission controller 39 applies a high voltage to each of the X-ray tube 13 and the X-ray tube 27. Based on an output of the high voltage applied by the X-ray emission controller 39, a dose of X-rays emitted from each of the X-ray tube 13 and the X-ray tube 27 as well as a timing of the X-ray emission are controlled.

The drive controller 41 individually controls a functional operation of each of the C-arm 17 and the C-arm 31. The drive controller 41 controls a drive mechanism (not illustrated) to appropriately control a direction and an amount of rotation of each of the C-arm 17 and the C-arm 31. A rotation angle of each of the C-arm 17 and the C-arm 31 is appropriately adjusted by the control of the drive controller 41. The rotation angles of the C-arm 17 and the C-arm 31 are detected by a rotation angle detector (not illustrated) such as a rotary encoder, and information of the rotation angles is transmitted to the host control unit 9 as required.

The rotation directions of the C-arm 17 and the C-arm 31 are represented as described below. As illustrated in FIG. 1, of directions of rotation along the axis of the body of the subject M, a direction of rotation toward a head is hereinafter denoted by “CRA” (Cranial), and a direction of rotation toward feet is hereinafter denoted by “CAU” (Caudal). As illustrated in FIG. 2, of directions of rotation around the axis of the body of the subject M, a direction of rotation to the left as viewed from the head is hereinafter denoted by “LAO” (Left Anterior Oblique), and a direction of rotation to the right as viewed from the head is hereinafter denoted by “RAO” (Right Anterior Oblique).

The rotation directions of the C-arm 17 and the C-arm 31 are represented by a combination of a direction (CRA or CAU) in which each C-arm rotates, of the directions of rotation along the axis of the body of the subject M, and a direction (LAO or RAO) in which each C-arm rotates, of the directions of rotation around the axis of the body of the subject M. The rotation angle of the C-arm 17 is represented by a combination of an angle at which the C-arm 17 rotates in the direction along the axis of the body of the subject M, and an angle at which the C-arm 17 rotates around the axis of the body of the subject M. The rotation angle of the C-arm 31 is also represented in the same manner as the rotation angle of the C-arm 17.

The image generator 43 is provided in a stage subsequent to the X-ray detector 15 and the X-ray detector 29. The image generator 43 performs various image processing based on an X-ray detection signal output from each of the X-ray detector 15 and the X-ray detector 29, and generates an X-ray image. In other words, the image generator 43 generates an X-ray image imaged with the first image pickup mechanism 5 and an X-ray image imaged with the second image pickup mechanism 7. The image generator 43 associates data of image information K with data of an X-ray image L to generate data of an information display image D. Details of the image information K and the information display image D will be described later.

The X-ray fluoroscopic imaging apparatus 1 further includes a storage unit 45 and a console 47. The storage unit 45 stores various information on the X-ray fluoroscopic imaging apparatus 1. The information stored in the storage unit 45 is, for example, information on X-ray imaging conditions such as a tube voltage and a tube current, and various information such as information on various X-ray images generated by the image generator 43 and on the image processing performed by the image generator 43. Examples of the storage unit 45 include a nonvolatile memory.

The console 47 is used to input an operator's instruction on an operation of the X-ray fluoroscopic imaging apparatus 1. The host control unit 9 performs integrated control according to the instruction input to the console 47 by a user. Examples of the console 47 include a keyboard input panel, a touch input panel, a mouse, a dial, a changeover switch, and a push button switch.

The image display device 11 includes an image display unit 49, an input unit 51, a layout storage unit 53, a layout setting unit 55, and an image display control unit 57. The image display unit 49 displays the information display image D generated by the image generator 43, namely, the X-ray image L to which the image information K is attached. Examples of the image display unit 49 include a liquid crystal monitor. The image display unit 49 may be, for example, suspended from a ceiling or mounted on a mobile trolley. In the present example, as illustrated in FIG. 1, the image display unit 49 is a large-screen monitor suspended from the ceiling.

The input unit 51 is used to input an operator's instruction on an operation of the image display device 11, and according to the instruction input to the input unit 51 by the user, various functional operations in the image display device 11 are executed. Examples of the input unit 51 include a keyboard input panel, a touch input panel, and a mouse.

The layout storage unit 53 stores information on a layout of images displayed on the image display unit 49. In Example 1, the layout storage unit 53 stores an arrangement pattern when a plurality of the information display images D are displayed on the image display unit 49. The layout storage unit 53 stores a positional relation between the X-ray image L and the image information K in each information display image D. The layout storage unit 53 stores a predetermined pattern (default pattern) in an initial state for an arrangement pattern of the plurality of information display images D and an arrangement pattern of the X-ray images L and the image information K in the information display images D.

The layout setting unit 55 receives the operator's instruction from the input unit 51, and sets anew a positional relation between the X-ray image L and the image information K in each information display image D according to the operator's instruction. In addition, the layout setting unit 55 sets anew an arrangement pattern of the plurality of information display images D displayed on a screen of the image display unit 49 according to the operator's instruction input from the input unit 51.

The image display control unit 57 controls a mode of displaying various image data on the image display unit 49. The image display control unit 57 controls the image display unit 49 to display each information display image D on the image display unit 49 according to an arrangement pattern set by the layout setting unit 55.

### <Configuration of Information Display Image>

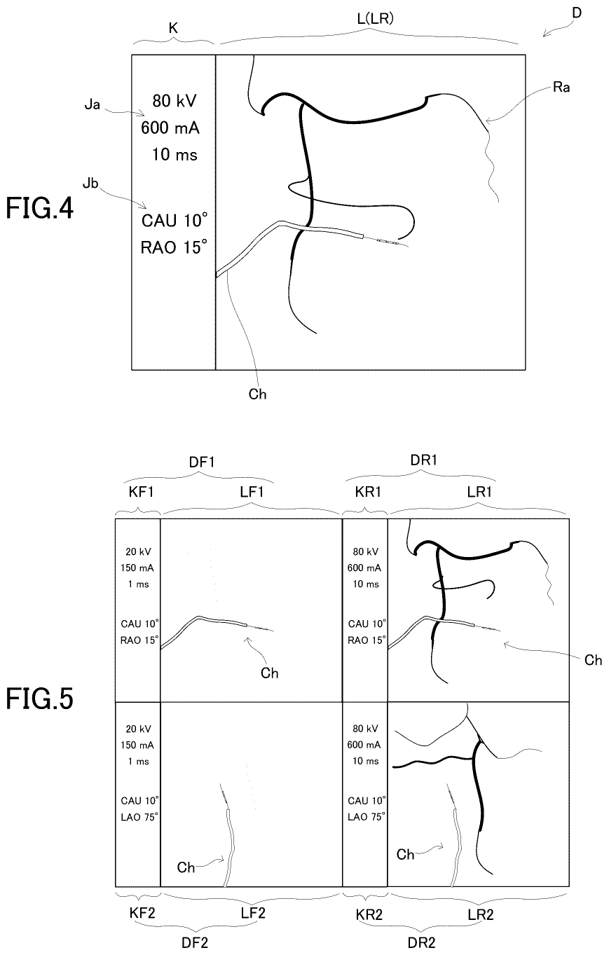

Next, a configuration of the information display image D displayed on the image display unit 49 will be described. As illustrated in FIG. 4, the information display image D includes an X-ray image L and image information K. The X-ray image L is an image showing an area of interest of the subject M, which is obtained by performing image processing appropriately on the X-ray detection signal output from the X-ray detector 15 or the X-ray detector 29. A region in which the X-ray image L is displayed in the information display image D corresponds to an X-ray image display region in the present example.

The image information K indicates information on the X-ray image L attached thereto. Examples of the information indicated by the image information K include information J1 on an emission condition and information J2on an emission direction. The information J1 on an emission condition is information on a condition under which X-rays are emitted. Examples of such information include information on an X-ray emission time in addition to the tube voltage or the tube current of the X-ray tube. The information J2 on an emission direction is information on a direction in which X-rays are emitted. Examples of such information include information on the rotation angle of each of the C-arm 17 and the C-arm 31. A region in which the image information K is displayed in the information display image D corresponds to an image information display region in the present example.

In the information display image D, the X-ray image L and the image information K are arranged in a row. FIG. 4 illustrates an arrangement pattern of the X-ray image L and the image information K in the initial state. In the initial state, it is determined that the region in which the image information K is arranged in the information display image D is located on the left of the region in which the X-ray image L is arranged. In Example 1, since the input unit 51 and the layout setting unit 55 are included, the arrangement pattern of the X-ray image L and the image information K in the information display image D can be changed. An operation of changing the arrangement pattern will be described later. The input unit 51 corresponds to an information display region change instruction unit in the present example. The layout setting unit 55 corresponds to an information display position setting unit in the present example.

FIG. 5 illustrates an arrangement pattern of information display images D when a plurality of X-ray images L are displayed on the image display unit 49. Two kinds of X-ray images L are obtained in angiographic imaging using a catheter Ch: one is obtained in a state in which a blood vessel is visualized with a contrast agent (hereinafter, referred to as a “reference image LR”) ; and the other is obtained in real time (hereinafter, referred to as an “acquired image LF”). The reference image LR and the acquired image LF are displayed on the image display unit 49, and a position of the catheter Ch is recognized while visually comparing the images.

In a case that angiographic imaging is performed with the biplane X-ray fluoroscopic imaging apparatus 1, the first image pickup mechanism 5 and the second image pickup mechanism 7 are used to image respective X-ray images L at different imaging angles to the subject M, and the X-ray images L are displayed on the image display unit 49. Specifically, an acquired image LF and a reference image LF imaged with the first image pickup mechanism 5 and an acquired image LF and a reference image LF imaged with the second image pickup mechanism 7, are displayed on the image display unit 49.

Note that the acquired image LF imaged with the first image pickup mechanism 5 is referred to as an acquired image LF1, and is distinguished from an acquired image LF2 that is the acquired image LF imaged with the second image pickup mechanism 7. In addition, the reference image LR imaged with the first image pickup mechanism 5 is referred to as a reference image LR1, and is distinguished from a reference image LR2 that is the reference image LR imaged with the second image pickup mechanism 7.

Respective pieces of image information K attached to the reference images FR1 and FR2 and the acquired images LF1 and LF2 are denoted by different reference signs of image information KR1, KR2, KF1, and KF2, and are individually distinguished. Of the information display images D, the reference image LR1 to which the image information KR1 is attached is referred to as an information display image DR1. Likewise, of the information display images D, the X-ray images L being the reference image LR2, the acquired image LF1, and the acquired image LF2 are distinguished respectively as an information display image DR2, an information display image DF1, and an information display image DF2.

FIG. 5 illustrates the arrangement pattern of the four information display images DR1, DR2, DF1, and DF2 in an initial state. The four information display images DR1, DR2, DF1, and DF2 are arranged in a row and a column in a matrix on the screen of the image display unit 49. In the arrangement pattern in the initial state according to Example 1, the information display images DF1 and DR1 obtained with the first image pickup mechanism 5 are arranged in an upper section, and the information display images DF2 and DR2 obtained with the second image pickup mechanism 7 are arranged in a lower section. The information display images DR1 and DR2 each including the reference image LR are arranged on the right of the information display images DF1 and DF2 each including the acquired image LF.

### <Overview of Functional Operation>

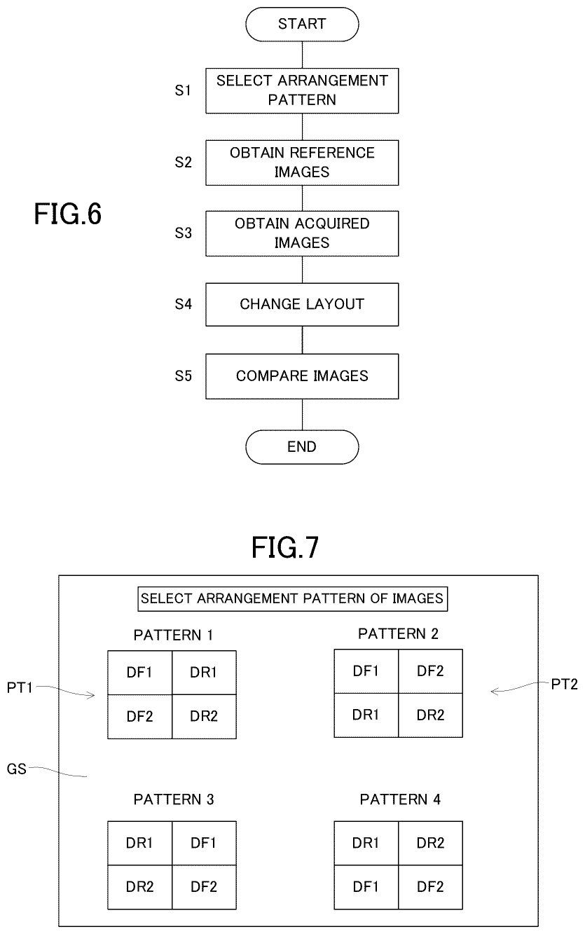

Next, a functional operation of the X-ray fluoroscopic imaging apparatus 1 according to Example 1 will be described. FIG. 6 is a flowchart illustrating the functional operation of the X-ray fluoroscopic imaging apparatus 1. In Example 1, a case that angiographic imaging is performed on a cardiovascular vessel by biplane imaging using the first image pickup mechanism 5 and the second image pickup mechanism 7, is taken as an example.

### Step S1 (Selection of Arrangement Pattern)

First, an arrangement pattern of a plurality of information display images D displayed on the image display unit 49 is selected before X-ray imaging is performed on the subject M. The user operates the console 47 to read arrangement patterns of the information display images D stored in the layout storage unit 53. The read arrangement patterns are displayed on a display GS included in the console 47, or the like, as illustrated in FIG. 7. In the arrangement patterns of the information display images D, as illustrated in FIG. 7, an arrangement pattern PT1 in which reference images LR are arranged on the right of acquired images LF, as well as an arrangement pattern PT2 in which reference images LR are arranged below acquired images LF, and the like are set in advance. The arrangement pattern PT1 is, in other words, an arrangement pattern of the information display images DR1, DR2, DF1, and DF2 as illustrated in FIG. 5.

The user selects a pattern suitable for a course of a medical operation from the arrangement patterns of the information display images D. Assume here that the arrangement pattern PT1 is selected as the arrangement pattern of the information display images D in the initial state.

### Step S2 (Obtainment of Reference Images)

Reference images are obtained after the arrangement pattern of the information display images D in the initial state is selected. Specifically, the user inserts the catheter Ch into the body of the subject M and allows the catheter Ch to reach a cardiovascular vessel of the subject M. After the catheter Ch reaches the cardiovascular vessel of a target area, the user rotates the C-arm 17 of the first image pickup mechanism 5 and the C-arm 31 of the second image pickup mechanism 7 to predetermined rotation angles, and then obtains contrast images as the reference images LR. The user administers a contrast agent to a blood vessel of the subject M from a tip of the catheter Ch.

After the administration of the contrast agent, imaging of X-ray images L is started. Specifically, an operator operates the console 47 to cause the X-ray tube 13 of the first image pickup mechanism 5 and the X-ray tube 27 of the second image pickup mechanism 7 to emit X-rays onto the subject M. The X-ray detector 15 detects the X-rays emitted from the X-ray tube 13 at a first imaging angle and transmitted through the area of interest of the subject M, and outputs an X-ray detection signal. The X-ray detector 29 detects the X-rays emitted from the X-ray tube 27 at a second imaging angle and transmitted through the area of interest, and outputs an X-ray detection signal.

The image generator 43 generates, as the reference image LR1, an X-ray image L showing a visualized blood vessel in the area of interest based on the X-ray detection signal output by the X-ray detector 15. The image generator 43 generates, as the reference image LR2, an X-ray image L showing a visualized blood vessel in the area of interest based on the X-ray detection signal output by the X-ray detector 29. The reference image LR1 and the reference image LR2 are X-ray images in which the same area of interest is imaged simultaneously from different imaging angles.

The image generator 43 generates an information display image D by attaching image information K to each of the reference image LR1 and the reference image LR2. Specifically, the image generator 43 generates the information display image DR1 by attaching the image information KR1 indicating information such as on the first imaging angle to the reference image LR1 imaged with the first image pickup mechanism 5. The image generator 43 generates also the information display image DR2 by attaching the image information KR2 indicating information such as on the second imaging angle to the reference image LR2 imaged with the second image pickup mechanism 7. Data of the generated information display image DR1 and information display image DR2 are transmitted to the image display device 11.

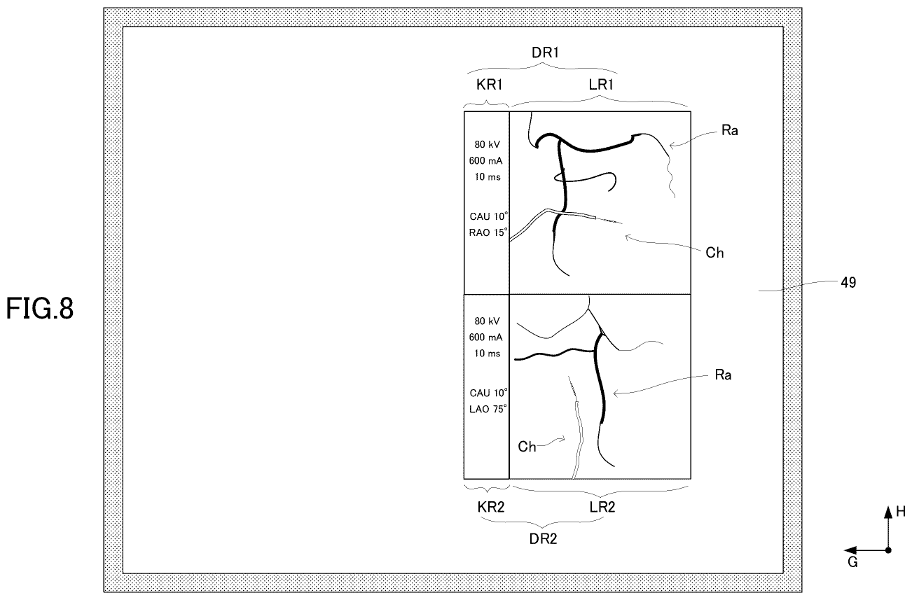

In the image display device 11, the image display control unit 57 controls the image display unit 49 to display the information display image DR1 and the information display image DR2 on the image display unit 49. Positions of the information display image DR1 and the information display image DR2 displayed on the image display unit 49, as illustrated in FIG. 8, are determined according to the arrangement pattern selected in step S1. Specifically, the information display image DR1 is displayed on an upper right part of the image display unit 49, and the information display image DR2 is displayed on a lower right part of the image display unit 49. The information display image DR1 and the information display image DR2 are arranged to be vertically adjacent.

As a time elapses after the contrast agent is administered to the blood vessel, the contrast agent spreads through the bloodstream, resulting in the contrast agent disappearing from the blood vessel in the area of interest. However, the reference image LR1 and the reference image LR2 obtained while the blood vessel is visualized are displayed on the image display unit 49, so that a position of a blood vessel Ra in the area of interest can be identified by referring to the reference image LR1 and the reference image LR2. The information display images D each including the reference image LR are obtained and then displayed on the image display unit 49. The procedure of step S2 is thus completed.

### Step S3 (Obtainment of Acquired Images)

The acquired images LF are obtained after the reference images LR are obtained. The user operates the console 47 again to cause each of the X-ray tube 13 and the X-ray tube 27 to emit X-rays onto the subject M at the imaging angles identical to those in step S2.

The image generator 43 generates, as the acquired image LF1, an X-ray image L showing a most recent area of interest based on an X-ray detection signal output by the X-ray detector 15. The image generator 43 generates, as the acquired image LF2, an X-ray image L showing a most recent area of interest based on an X-ray detection signal output by the X-ray detector 29. The acquired image LF1 and the acquired image LF2 are X-ray images of the same area of interest imaged simultaneously from different imaging angles, and show most recent images of the catheter Ch, respectively.

The image generator 43 generates an information display image D by attaching image information K to each of the acquired image LF1 and the acquired image LF2. Specifically, the image generator 43 generates the information display image DF1 by attaching the image information KF1 indicating the information such as on the first imaging angle to the acquired image LF1 imaged with the first image pickup mechanism 5. The image generator 43 generates also the information display image DF2 by attaching the image information KF2 indicating the information such as on the second imaging angle to the acquired image LF2 imaged with the second image pickup mechanism 7. Data of the generated information display image DF1 and information display image DF2 are transmitted to the image display device 11 from the image generator 43.

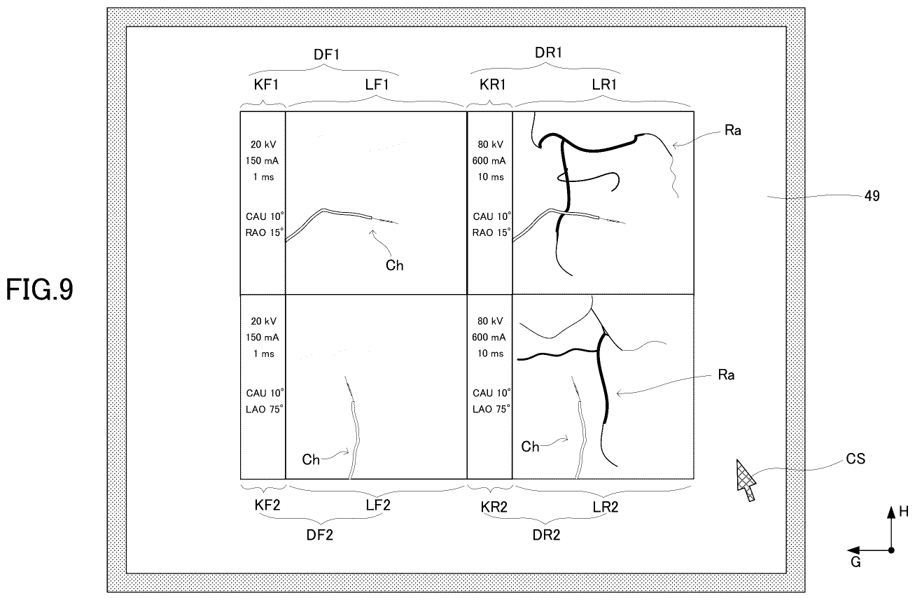

In the image display device 11, the image display control unit 57 controls the image display unit 49 to display the information display image DF1 and the information display image DF2 on the image display unit 49. Positions of the information display image DF1 and the information display image DF2 displayed on the image display unit 49, as illustrated in FIG. 9, are determined according to the arrangement pattern selected in step S1. Specifically, the information display image DF1 is displayed adjacently to the left of the information display image DR1, and the information display image DF2 is displayed adjacently to the left of the information display image DR2.

In order to catch a more recent state of the catheter Ch, the user intermittently obtains acquired images LF. Specifically, X-rays are intermittently emitted onto the subject M from each of the X-ray tube 13 and the X-ray tube 27. Every time the X-rays are emitted, the image generator 43 generates an information display image DF1 and an information display image DF2 anew. That is, an information display image DF1 and an information display image DF2 showing a real-time catheter Ch are displayed on the image display unit 49. The information display images D each including the acquired image LF are obtained and then displayed on the image display unit 49. The procedure of step S3 is thus completed.

### Step S4 (Change of Layout)

In the initial state, it is determined that the X-ray image L is arranged on the right of the image information K in each of the information display images D. Hence, at the time of completing step S3, the acquired image LF1 is arranged on the right of the image information KF1 in the information display image DF1, and the reference image LR1 is arranged on the right of the image information KR1 in the information display image DR1. When the information display images DR1, DR2, DF1, and DF2 are displayed on the image display unit 49 as in the arrangement pattern in the initial state, the image information KR1 is arranged between the acquired image LF1 and the reference image LR1. The image information KR2 also is arranged between the acquired image LF2 and reference image LR2.

When performing catheter manipulation in angiographic imaging, the user needs to move the eyes alternately to the reference image LR being the contrast image and the acquired image LF being a real-time image without the contrast agent for visual comparison. At this time, the image information K arranged between the reference image LR and the acquired image LF becomes an obstacle to an operation of visual comparison. Thus, in Example 1, a layout of the information display image DR1 is changed from the initial state by a user's operation. Specifically, the user performs an operation of changing a positional relation between the image information KR1 and the reference image LR1 to make the acquired image LF1 and the reference image LR1 adjacent.

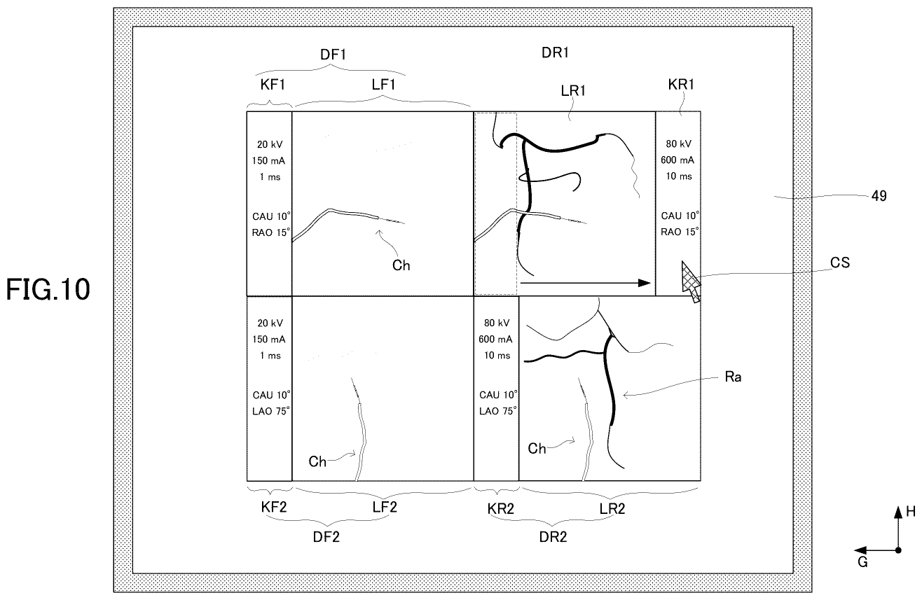

When changing the layout of the information display image DR1, the user operates the input unit 51 included in the image display device 11 to input an instruction to arrange the acquired image LF1 on the right of the reference image LR1. As illustrated in FIG. 10, for example, a cursor CS is operated on the screen of the image display unit 49 with a mouse or the like to select a region of the image information KF1. Then, an operation of dragging the image information KF1 in the right direction is performed with a mouse.

The instruction input by the user using the input unit 51 is received by the layout setting unit 55. The layout setting unit 55 then changes the arrangement pattern of the image information K and the X-ray image L in the information display image D according to a content of the instruction. Specifically, the information display image DR1 is changed from the arrangement pattern in which the image information KR1 is arranged on the left of the reference image LR1 in the initial state, to an arrangement pattern in which the image information KR1 is arranged on the right of the reference image LR1.

The layout setting unit 55 transmits, to the layout storage unit 53 and the image display control unit 57, the arrangement pattern of the image information KR1 and the reference image LR1 after the change, that is, the information on the layout of the information display image DR1 after the change. The layout storage unit 53 stores a layout setting of the information display image DR1 after the change.

The image display control unit 57 controls the image display unit 49 to change a mode of displaying the information display image DR1 on the image display unit 49 according to the information on the layout of the information display image DR1 set anew by the layout setting unit 55. The layout of the information display image DR1 displayed on the image display unit 49 is changed by the control of the image display control unit 57. Specifically, as illustrated in FIG. 10, the image information KR1 is displayed on the right of the reference image LR1 in the information display image DR1. As a result, the X-ray image L (reference image LR1) in the information display image DR1 and the X-ray image L (acquired image LF1) in the information display image DF1 are adjacent in a left-right direction G.

Changing the layout also results in sides of the reference image LR1 and the acquired image LF1 across the direction (G direction) in which the images are arranged in the row, being flush with each other. In other words, a position (height) of the reference image LR1 and a position (height) of the acquired image LF1 coincide with each other in a direction (H direction) orthogonal to the row direction. By changing the layout such that the heights of the reference image LR1 and the acquired image LF1 coincide with each other, the user can compare the reference image LR1 and the acquired image LF1 while moving the eyes parallel with the G direction. Accordingly, when a position of an image shown in the reference image LR1 and a position of an image shown in the acquired image LF1 are compared and examined, there is no need to take into account the difference in height between these images while moving the eyes in an oblique direction, which eases a motion in comparing the reference image LR1 and the acquired image LF1.

After the layout of the information display image DR1 is changed such that the reference image LR1 and the acquired image LF1 are adjacent, an operation of changing the layout of the information display image DR2 is performed. That is, in order to change a positional relation between the image information KR2 and the reference image LR2, in which the image information KR2 is arranged between the acquired image LF2 and the reference image LR2 in the layout in the initial state, the layout of the information display image DR2 is changed.

As an example, the user inputs an instruction to arrange the image information KR2 on the right of the reference image LR2 by performing an operation of dragging the image information KF2 in the right direction while the image information KF2 is selected. The user's operation is received by the layout setting unit 55. Then, the layout of the information display image DR2 is changed from the pattern in which the image information KR2 is arranged on the left of the reference image LR2 to a pattern in which the image information KR2 is arranged on the right of the reference image LR2.

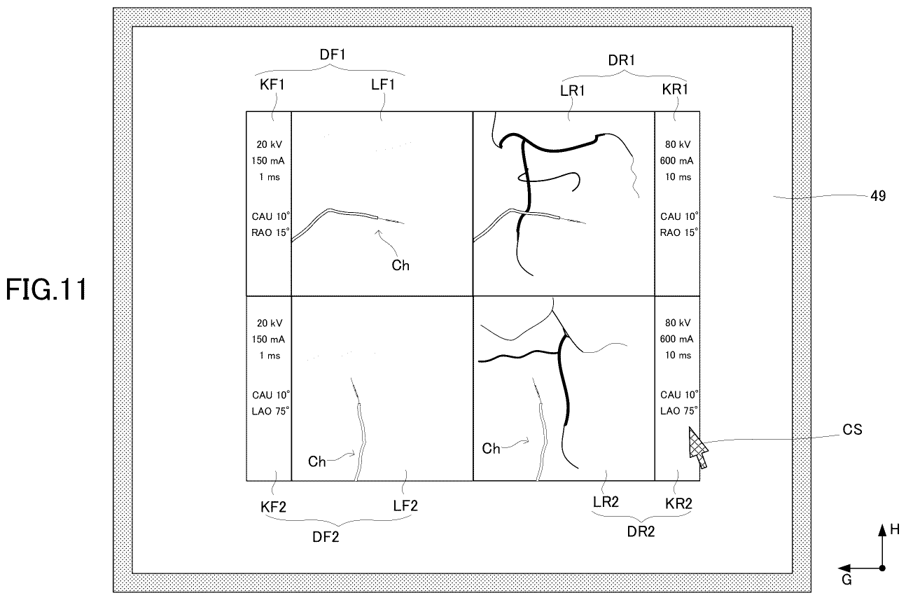

The image display control unit 57 causes the image display unit 49 to display the information display image DR2 in conformity with the layout setting after the change. Thus, as illustrated in FIG. 11, the image information KR2 is displayed on the right of the reference image LR2 in the information display image DR2. As a result, the X-ray image L (reference image LR2) in the information display image DR2 and the X-ray image L (acquired image LF2) in the information display image DF2 are adjacent in the left-right direction G. Sides of the reference image LR2 and the acquired image LF2 across the direction (G direction) in which the images are arranged in the row, are flush with each other.

In this manner, the layout of the information display images D is appropriately changed so as to facilitate the comparison between the reference image LR and the acquired image LF. In Example 1, the layout of the information display image DR1 and the information display image DR2 are changed. The procedure of step S4 is thus completed.

### Step S5 (Comparison of Images)

After the layout of the information display images D is changed, the medical operation is continued while the reference image LR and the acquired image LF are compared. The user identifies the real-time position of the catheter Ch shown in the acquired image LF and the position of the visualized blood vessel Ra shown in the reference image LR, and recognizes a positional relation between the catheter Ch and the blood vessel Ra in the area of interest of the subject M.

Specifically, the user directs the eyes alternately to the reference image LR1 and the acquired image LF1 displayed on the image display unit 49 for visual comparison, and recognizes the positional relation between the catheter Ch and the blood vessel Ra in the area of interest of the subject M in a first imaging direction. Moreover, the user visually compares the reference image LR2 and the acquired image LF2, and recognizes the positional relation between the catheter Ch and the blood vessel Ra in the area of interest in a second imaging direction. The user operates the catheter Ch while recognizing the accurate positional relation between the catheter Ch and the blood vessel Ra, and continues medical treatment.

In Example 1, the user visually compares the reference image LR and the acquired image LF, with the layout of the information display images D changed. In the initial state, the reference image LR and the acquired image LF are opposite to each other with the image information K interposed therebetween, but the operation of changing the layout allows the reference image LR and the acquired image LF being targets of comparison to be adjacent.

Thus, it is possible to avoid elements of the image information K becoming the obstacle to the comparison and examination for the user when the eyes are directed alternately to the reference image LR and the acquired image LF being the targets of comparison. Since the reference image LR and the acquired image LF are adjacent, a distance over which the user moves the eyes is shortened by a width of the image information K. Accordingly, it is possible to alleviate a fatigue experienced by the user when comparing the reference image LR and the acquired image LF.

Positions (heights) of the acquired image LF and the reference image LR arranged adjacently in the left-right direction G coincide with each other in the up-down direction H. Hence, while moving the eyes left and right alternately, the user can compare the acquired image LF and the reference image LR without taking into account a positional difference of the images in the up-down direction H.

During the ongoing medical operation, the user enables comparison of the reference image LR and the acquired image LF, as well as comparison of the acquired image LF1 and the acquired image LF2 that are imaged from different directions. Thus, the user three-dimensionally recognizes a position of the catheter Ch and a structure of the area of interest of the subject M by visually comparing the acquired image LF1 and the acquired image LF2 arranged adjacently in the up-down direction H.

Since the image information K is not arranged between the acquired image LF1 and the acquired image LF2, it is possible to avoid the image information K becoming the obstacle when the user moves the eyes alternately to the acquired image LF1 and the acquired image LF2.

Positions of the acquired image LF1 and the acquired image LF2 arranged adjacently in the up-down direction H coincide with each other in the left-right direction G. Hence, while moving the eyes up and down alternately, the user can compare the acquired image LF1 and the acquired image LF2 without taking into account a positional difference of the images in the left-right direction G. The user completes the operation of the catheter Ch using each of the information display images D with the layout changed, and a series of processes for the angiographic imaging using the X-ray fluoroscopic imaging apparatus 1 is finished.

### Example 2

Next, Example 2 of the present invention will be described. In Example 1, the layout of the information display images D is changed after the X-rays are emitted onto the subject M. That is, the operation of changing the layout of the information display images D each including the reference image LR or the acquired image LF, is performed while the generated reference image LR and acquired image LF being targets of comparison are displayed on the image display unit 49. Note that an X-ray fluoroscopic imaging apparatus 1 according to Example 2 has a configuration common to that of Example 1, and detailed description of the configuration is omitted.

In Example 2, a layout of information display images D is changed in advance before X-rays are emitted onto a subject M. Specifically, an operation of changing a layout, stored in a layout storage unit 53, of a plurality of information display images D is performed beforehand for an arrangement pattern of the information display images D to be displayed on an image display unit 49.

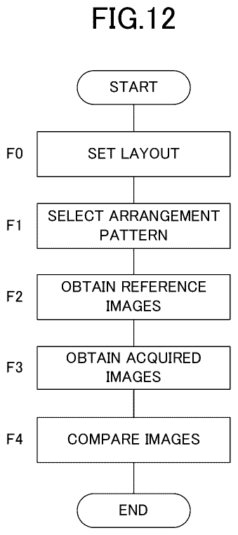

A functional operation of the X-ray fluoroscopic imaging apparatus 1 according to Example 2 will be described. FIG. 12 is a flowchart illustrating the functional operation of the X-ray fluoroscopic imaging apparatus 1 according to Example 2. In Example 2, a case that angiographic imaging is performed on a cardiovascular vessel by biplane imaging, is taken as an example in the Same manner as in Example 1.

### Step F0 (Setting of Layout)

In Example 2, as a stage previous to the angiographic imaging, an operation of setting a layout is performed for a pattern in which the plurality of information display images D are arranged on a screen of the image display unit 49. In an image display device 11, the layout storage unit 53 stores arrangement patterns PT1, PT2, and the like of the information display images D as illustrated in FIG. 7.

The user operates a console 47 to read the arrangement patterns of the information display images D stored in the layout storage unit 53. The read arrangement patterns are displayed on a display GS or the like, as illustrated in FIG. 7.

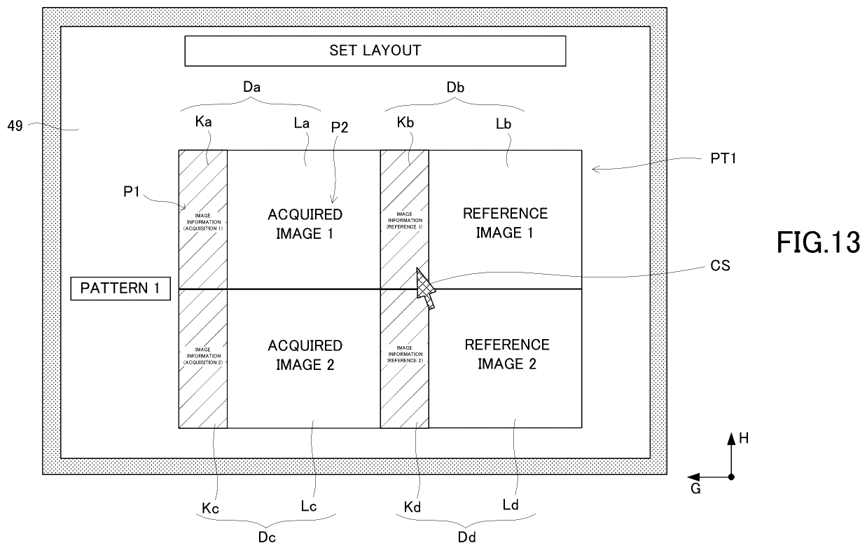

After reading the arrangement patterns of the information display images D, the user selects an arrangement pattern as a target of layout setting operation. Assume here that the arrangement pattern PT1 is selected as a layout setting target. Information of the selected arrangement pattern PT1 is read from the layout storage unit 53, and images of which the layout in the arrangement pattern PT1 is changed are displayed on a display screen of the image display unit 49, or the like, as illustrated in FIG. 13.

As illustrated in FIG. 13, in the arrangement pattern PT1, four information display images D are arranged to be aligned in two rows in the left-right direction G and in two columns in the up-down direction H. In other words, arrangement regions Da, Db, Dc, and Dd occupy an upper left portion, an upper right portion, a lower left portion, and a lower right portion of an entire region of the arrangement pattern PT1. The arrangement regions Da, Db, Dc, and Dd are regions to arrange information display images DR1, DR2, DF1, and DF2.

The arrangement region Da contains an arrangement region La and an arrangement region Ka, and the arrangement region Ka is located on the left of the arrangement region La. The arrangement region La is a region to arrange an acquired image LF1, and the arrangement region Ka is a region to arrange image information KF1. As illustrated in FIG. 13, information P1 is provided in the arrangement region La, and information P2 is provided in the arrangement region Ka, on the screen of the image display unit 49. The information P1 is information visually indicating the region to arrange the acquired image LF1. The information P2 is information visually indicating the region to arrange the image information KF1.

The arrangement region Db contains an arrangement region Lb and an arrangement region Kb. The arrangement region Lb is a region to arrange a reference image LR1, and the arrangement region Kb is a region to arrange image information KR1. The arrangement region Dc contains an arrangement region Lc and an arrangement region Kc. The arrangement region Lc is a region to arrange an acquired image LF2, and the arrangement region Kc is a region to arrange image information KF2. The arrangement region Dd contains an arrangement region Ld and an arrangement region Kd. The arrangement region Ld is a region to arrange a reference image LR2, and the arrangement region Kd is a region to arrange image information KR2. Similarly to the arrangement region La and the arrangement region Ka, information for visually identifying an arranged X-ray image or image information is provided in each of the arrangement regions Lb to Ld and the arrangement regions Kb to Kd.

A user looks over the display screen of the image display unit 49 as illustrated in FIG. 13 and recognizes a position at which each of the reference image LR and the acquired image LF is arranged and a position at which each piece of the image information K is arranged. The user recognizes that when starting angiographic imaging in an initial state of the arrangement pattern PT1, the image information KR1 is arranged between the reference image LR1 and the acquired image LF1 intended for visual comparison, and that the image information KR1 becomes an obstacle to a motion in visual comparison.

Thus, as a stage previous to the angiographic imaging, the user performs an operation of changing the layout of the arrangement pattern PT1. Specifically, the user first performs an operation of changing the layout of the arrangement pattern PT1 such that the arrangement region La being the region to display the acquired image LF1 and the arrangement region Lb being the region to display the reference image LR1, are adjacent.

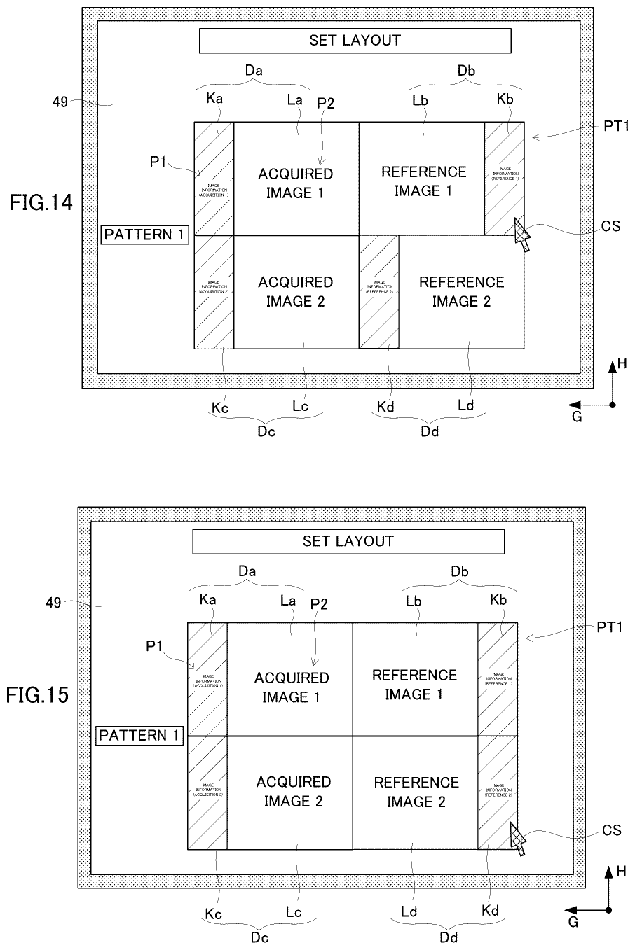

More specifically, the user first performs the operation of placing a cursor CS on the arrangement region Kb being the region to display the image information KR1, moving the arrangement region Kb in the right direction, and then changing the layout of the arrangement region Db so as to locate the arrangement region Kb on the right of the arrangement region Lb, as illustrated in FIG. 14. This changing operation results in locating the arrangement region Kb on the right of the arrangement region Lb while locating the arrangement region Ka on the left of the arrangement region Lb. Thus, the layout of the arrangement pattern PT1 is changed to arrange the arrangement region La and the arrangement region Lb adjacently in the left-right direction.

The user also recognizes that in the initial state of the arrangement pattern PT1, the image information KR2 is arranged between the reference image LR2 and the acquired image LF2 intended for visual comparison, and that the image information KR2 becomes the obstacle to the motion in visual comparison. Thus, the user performs an operation of placing the cursor CS on the arrangement region Kd and moving the arrangement region Kd in the right direction to change the layout of the arrangement region Dd so as to locate the arrangement region Kd on the right of the arrangement region Ld, as illustrated in FIG. 15. Consequently, the layout of the arrangement pattern PT1 is changed to arrange the arrangement region Lc and the arrangement region Ld adjacently in the left-right direction.

After the operation of changing the layout of the arrangement pattern PT1 from the initial state is completed, the user saves the changed layout. Through this saving operation, the layout storage unit 53 stores the arrangement pattern PT1 with the layout changed. Storing the changed layout enables the operation of changing the layout of the arrangement pattern PT1 to be omitted for a next time the angiographic imaging is performed with the arrangement pattern PT1. The layout of the arrangement pattern PT1 is changed. The process of step F0 is thus completed.

### Step F1 (Selection of Arrangement Pattern)

After the operation of changing the layout is completed, the angiographic imaging is started. Similarly to step S1 according to Example 1, an arrangement pattern of a plurality of information display images D displayed on the image display unit 49 is selected. The user operates the console 47 to read arrangement patterns (FIG. 7) of the information display images D stored in the layout storage unit 53. Then, the arrangement pattern PT1 is selected from the read arrangement patterns and used.

In step F0, the layout of the arrangement pattern PT1 is changed and the arrangement pattern PT1 with the layout changed is stored in the layout storage unit 53. The arrangement pattern read from the layout storage unit 53 is therefore an arrangement as illustrated in FIG. 15. Specifically, the arrangement region La and the arrangement region Lb are arranged adjacently in the left-right direction, and the arrangement region Lc and the arrangement region Ld are arranged adjacently in the left-right direction.

### Step F2 (Obtainment of Reference Images)

Reference images are obtained after the arrangement pattern of the information display images D in the initial state is selected. The process of step F2 according to Example 2 is similar to the process of step S2 according to Example 1. Specifically, the user inserts a catheter Ch into a body of the subject M and allows the catheter Ch to reach a cardiovascular vessel of the subject M. After the catheter Ch reaches the cardiovascular vessel of a target area, the user rotates a C-arm 17 of a first image pickup mechanism 5 and a C-arm 31 of a second image pickup mechanism 7 to predetermined rotation angles, and then obtains contrast images as reference images LR. The user administers a contrast agent to a blood vessel of the subject M from a tip of the catheter Ch.

After the administration of the contrast agent, imaging of X-ray images L is started. Specifically, an operator operates the console 47 to cause an X-ray tube 13 of the first image pickup mechanism 5 and an X-ray tube 27 of the second image pickup mechanism 7 to emit X-rays onto the subject M. An X-ray detector 15 detects the X-rays emitted from the X-ray tube 13 at a first imaging angle and transmitted through an area of interest of the subject M, and outputs an X-ray detection signal. An X-ray detector 29 detects the X-rays emitted from the X-ray tube 27 at a second imaging angle and transmitted through the area of interest, and outputs an X-ray detection signal.

An image generator 43 generates a reference image LR1 based on the X-ray detection signal output by the X-ray detector 15, and generates a reference image LR2 based on the X-ray detection signal output by the X-ray detector 29. The image generator 43 generates also an information display image DR1 by attaching image information KR1 to the reference image LR1. The image generator 43 generates an information display image DR2 by attaching image information KR2 to the reference image LR2. Data of the generated information display image DR1 and information display image DR2 are transmitted to the image display device 11.

In the image display device 11, an image display control unit 57 controls the image display unit 49 to display the information display image DR1 and the information display image DR2 on the image display unit 49. Positions of the information display image DR1 and the information display image DR2 displayed on the image display unit 49 are determined according to the arrangement pattern PT1 read in step F1.

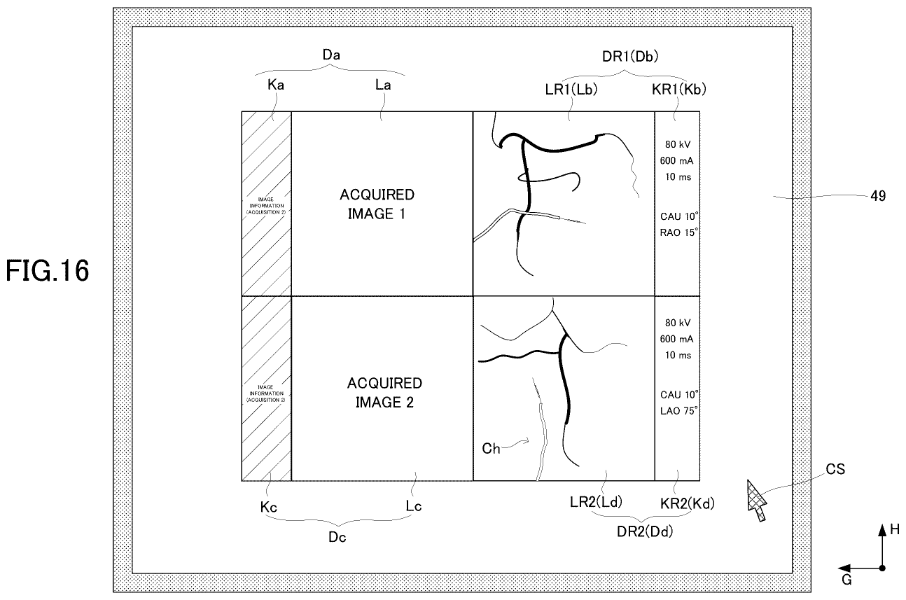

That is, as illustrated in FIG. 16, the information display image DR1 is displayed in the display region Db after the layout change. Specifically, the reference image LR1 is displayed at a position of the display region Lb, and the image information KR1 is displayed on the right (the position of the display area Kb) of the reference image LR1. The information display image DR2 is displayed in the display region Dd after the layout change. Specifically, the reference image LR2 is displayed at a position of the display region Ld, and the image information KR2 is displayed on the right (the position of the display area Kd) of the reference image LR2. The information display images DR1 and DR2 are obtained and then displayed on the image display unit 49. The procedure of step S2 is thus completed.

### Step F3 (Obtainment of Acquired Images)

Acquired images LF are obtained after the reference images LR are obtained. The process of step F3 according to Example 2 is similar to the process of step S3 according to Example 1. The user operates the console 47 again to cause each of the X-ray tube 13 and the X-ray tube 27 to emit X-rays onto the subject M at the imaging angles identical to those in step F2.

The image generator 43 generates, as an acquired image LF1, an X-ray image L showing a most recent area of interest based on an X-ray detection signal output by the X-ray detector 15. The image generator 43 generates, as an acquired image LF2, an X-ray image L showing a most recent area of interest based on an X-ray detection signal output by the X-ray detector 29. The image generator 43 generates an information display image DF1 by attaching image information KF1 to the acquired image LF1. The image generator 43 generates also an information display image DF2 by attaching image information KF2 to the acquired image LF2. Data of the generated information display image DF1 and information display image DF2 are transmitted to the image display device 11 from the image generator 43.

In the image display device 11, the image display control unit 57 controls the image display unit 49 to display the information display image DF1 and the information display image DF2 on the image display unit 49. Positions of the information display image DF1 and the information display image DF2 displayed on the image display unit 49 are determined according to the arrangement pattern PT1 read in step F1.

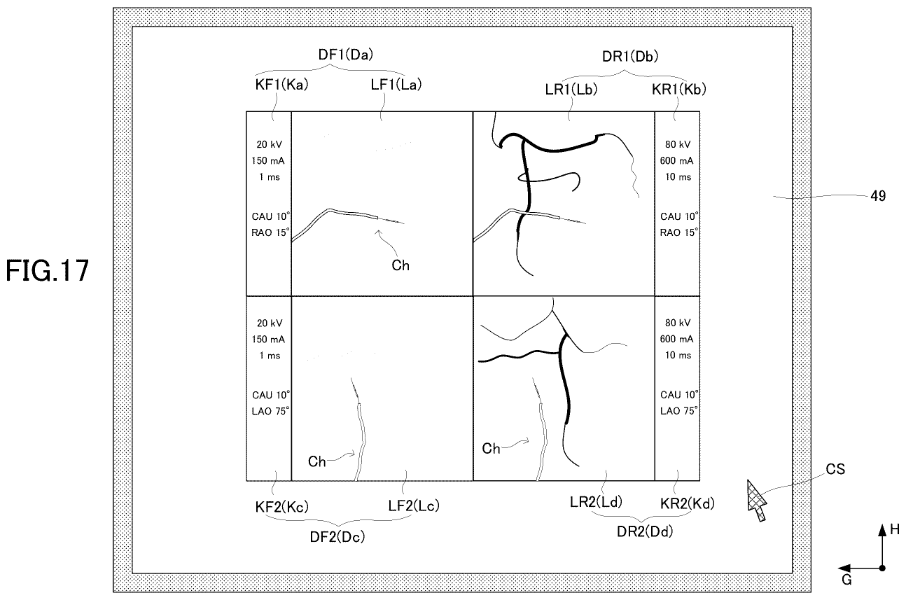

That is, as illustrated in FIG. 17, the information display image DF1 is displayed in the display region Da after the layout change. Specifically, the acquired image LF1 is displayed at a position of the display region La, and the image information KF1 is displayed on the left (the position of the display area Ka) of the acquired image LF1. The information display image DF2 is displayed in the display region Dc after the layout change. Specifically, the acquired image LF2 is displayed at a position of the display region Lc, and the image information KF2 is displayed on the left (the position of the display area Kc) of the acquired image LF2.

As a result, the acquired image LF1 and the reference image LR1 imaged with the first image pickup mechanism 5 are displayed adjacently in the left-right direction G. Similarly, the acquired image LF2 and the reference image LR2 imaged with the second image pickup mechanism 7 are displayed adjacently in the left-right direction G. In this manner, by performing the process of step F0 in advance, the reference images LR generated in step F2 and the acquired images generated in step F3 are automatically displayed at the positions adjacent to each other.

In order to catch a more recent state of the catheter Ch, the user intermittently obtains acquired images LF. Every time the X-rays are emitted, the image generator 43 generates an information display image DF1 and an information display image DF2 anew and the generated images are displayed on the image display unit 49. The information display image DF1 and the information display image DF2 each including the acquired image LF are displayed on the image display unit 49. The procedure of step S3 is thus completed.

### Step F4 (Comparison of Images)

After each of the reference images LR and each of the acquired images LF are displayed on the image display unit 49, a medical operation is continued while the reference image LR and the acquired image LF are compared. Step F4 according to Example 2 is a process common to the process of step S5 according to Example 1. The user identifies a real-time position of the catheter Ch shown in the acquired image LF and a position of a visualized blood vessel Ra shown in the reference image LR, and recognizes a positional relation between the catheter Ch and the blood vessel Ra in the area of interest of the subject M.

The user directs the eyes alternately to the reference image LR1 and the acquired image LF1 displayed on the image display unit 49 for visual comparison, and recognizes the positional relation between the catheter Ch and the blood vessel Ra in the area of interest of the subject M in a first imaging direction. Moreover, the user visually compares the reference image LR2 and the acquired image LF2, and recognizes the positional relation between the catheter Ch and the blood vessel Ra in the area of interest in a second imaging direction. The user operates the catheter Ch while recognizing the accurate positional relation between the catheter Ch and the blood vessel Ra, and continues medical treatment.

In Example 2, the user visually compares the reference image LR and the acquired image LF, with the layout of the information display images D changed, in the Same manner as in Example 1. By performing the operation of changing the layout, the reference image LR and the acquired image LF being targets of comparison are adjacent. Thus, it is possible to avoid elements of the image information K becoming the obstacle to the comparison and examination for the user when the eyes are directed alternately to the reference image LR and the acquired image LF being the targets of comparison. Accordingly, it is possible to alleviate a fatigue experienced by the user when comparing the reference image LR and the acquired image LF.

Moreover, in Example 2, since the layout of the information display images D is changed in advance in step F0 before the X-ray imaging is started, the reference image LR and the acquired image LF are already displayed adjacently when the reference image LR and the acquired image LF are displayed on the image display unit 49. There is therefore no need to perform the operation of changing the layout during an ongoing medical operation, so that a time required for the medical operation can be further reduced. The user completes the operation of the catheter Ch using each of the information display images D with the layout changed, and a series of processes for the angiographic imaging using the X-ray fluoroscopic imaging apparatus 1 is finished.

### <Advantageous Effects of Configuration According to Embodiment>

(Item 1) An X-ray fluoroscopic imaging apparatus according to the present embodiment includes: an X-ray tube that emits an X-ray to a subject; an X-ray detector that detects an X-ray transmitted through the subject; an arm that supports the X-ray tube and the X-ray detector such that the X-ray tube and the X-ray detector are opposite to each other; an arm rotation mechanism that rotates the arm around a predetermined axis; an X-ray image generator that generates an X-ray image using a detection signal output by the X-ray detector; and an X-ray image display device that displays the X-ray image. The X-ray image display device includes: an image display unit that is capable of displaying, in a row and a column in a matrix, information display images each containing an X-ray image display region in which the X-ray image showing a predetermined area of interest is displayed and an image information display region in which information on the X-ray image is displayed; an information display region change instruction unit that inputs an instruction to change a position of the image information display region in each of the information display images displayed in the row and the column on the image display unit, such that at least two of the X-ray image display regions are displayed adjacently; and an image display control unit that controls the image display unit such that based on a content of the instruction input to the information display region change instruction unit, the position of the image information display region in the information display image is changed to display the X-ray image display regions adjacently.

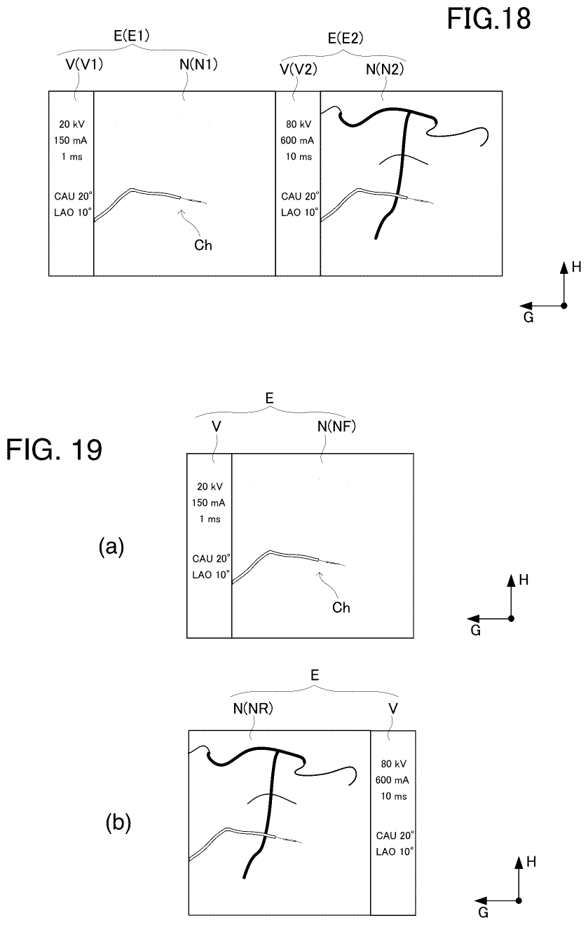

An advantageous effect of the X-ray fluoroscopic imaging apparatus according to Item 1 will be described with reference to FIGS. 18 to 21. In conventional X-ray fluoroscopic imaging apparatuses, a positional relation between an X-ray image N and image information V is unchanged in an information display image E with the image information V attached to the X-ray image N. In a case that the X-ray image N is always located on the right of the image information V in the information display image E, for example, when two information display images E are horizontally arranged in a row, the image information V becomes an obstacle to a motion in visually comparing X-ray images N. Specifically, when an X-ray image N1 (for example, an acquired image) included in an information display image E1 and an X-ray image N2 (for example, a reference image) included in an information display image E2 are observed for comparison, image information V2 included in the information display image E2 becomes the obstacle as illustrated in FIG. 18.

As a method of avoiding the problem as illustrated in FIG. 18, use of a configuration of a comparative example is conceivable, the configuration in which the positional relation between the X-ray image N and the image information V in the information display image E is made different between a case of the X-ray image N being an acquired image NF and a case of the X-ray image N being a reference image NR. In the comparative example, as illustrated in FIG. 19(a), when the X-ray image N is the acquired image NF, the X-ray image N is positioned to be always arranged on the left of the image information V in the information display image E. When the X-ray image N is the reference image NR, on the other hand, as illustrated in FIG. 19(b), the X-ray image N is positioned to be always arranged on the right of the image information V in the information display image E.

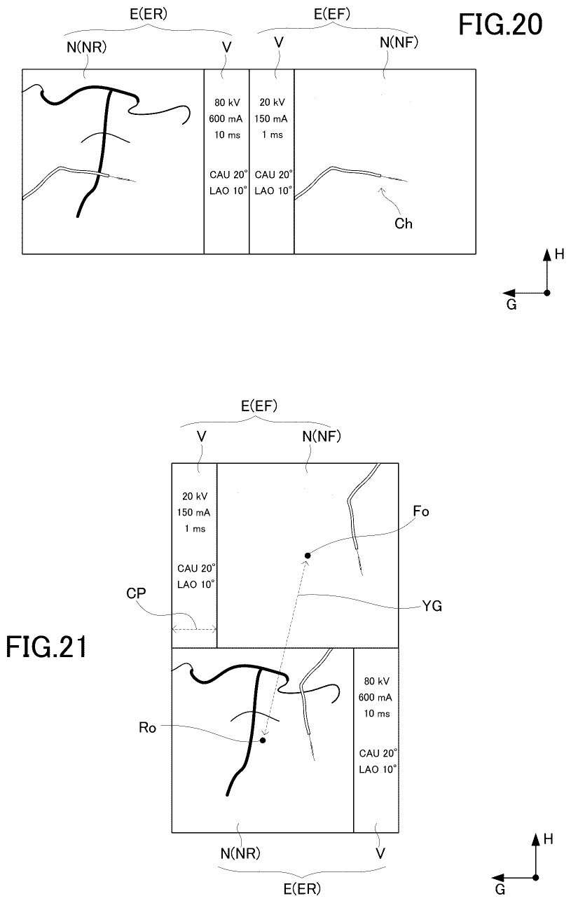

This comparative example enables the problem as illustrated in FIG. 18 to be avoided when the information display image E (information display image EF) including the acquired image NF is arranged adjacently to the left of the information display image E (information display image ER) including the reference image NR, as illustrated in FIG. 20. That is, the information display image EF is arranged on the left of the information display image ER, and the acquired image NF and the reference image NR are horizontally adjacent. Thus, it is possible to avoid the image information V becoming the obstacle to the motion in comparing the acquired image NF and the reference image NR.

With this comparative example, however, it is difficult to surely avoid the problem as illustrated in FIG. 18. A desirable arrangement pattern of the acquired image NF and the reference image NR for observing and comparing the acquired image NF and the reference image NR, differs between users. That is, some users desire to arrange the acquired image NF on the left of the reference image NR for visual comparison, whereas some other users desire to arrange the acquired image NF above the reference image NR for visual comparison.

In the comparative example having the configurations of FIG. 19(a) and 19(b), the problem that the image information V becomes the obstacle to the comparison and examination can be avoided only when the information display image EF is arranged on the left of the information display image ER. As illustrated in FIG. 21, in a case that the information display image EF is arranged on the right of the information display image ER, two pieces of the image information V are present between the acquired image NF and the reference image NR, which becomes more awkward in motion in comparing the acquired image NF and the reference image NR.

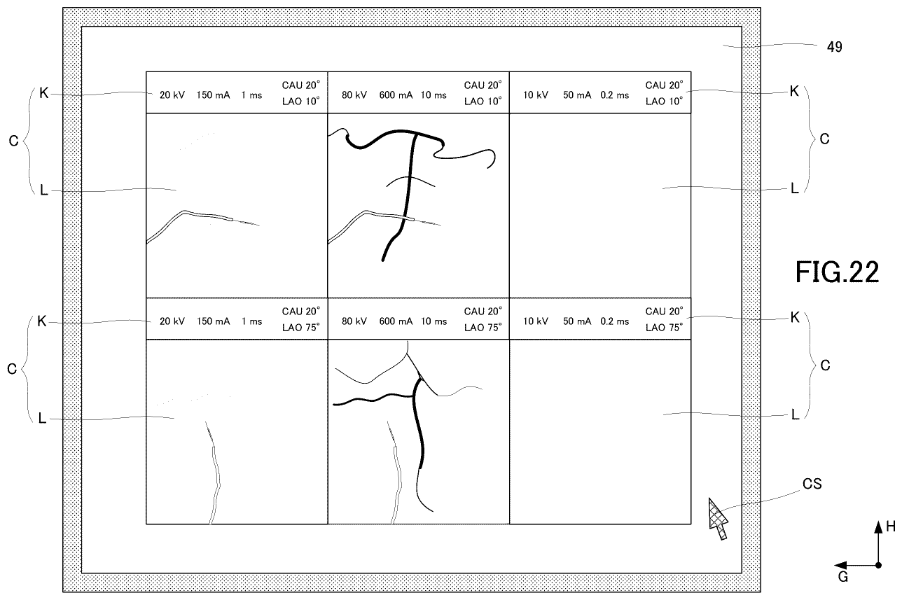

Further, in the comparative example, vertically arranging the information display image EF and the information display image ER produces a new problem. As illustrated in FIG. 22, when the information display image EF is arranged above the information display image ER, the positions of the information display image EF and the information display image ER are aligned in the left-right direction G. However, the information display image EF has the image information V arranged on the left of the acquired image NF, whereas the information display image ER has the image information V arranged on the right of the reference image NR.

As a result, a positional difference arises in the acquired image NF and the reference image NR in the left-right direction G, and thus the user needs to constantly move the eyes in an oblique direction for visually comparing the acquired image NF and the reference image NR. For example, when comparing a central area Fo of the information display image EF and a central area Ro of the information display image ER, the user needs to constantly move the eyes from one area to the other in the oblique direction along a trajectory indicated by an arrow YG in FIG. 21, and compare elements of the images while compensating for an amount of misregistration CP in the left-right direction G between the acquired image NF and the reference image NR in the brain. There is a concern that the motion in visually comparing the acquired image NF and the reference image NR that are obliquely different in position as illustrated in FIG. 20, increases a burden imposed on the user more than the motion in visually comparing the acquired image NF and the reference image NR that are only vertically or horizontally different in position.

To address such a conventional configuration, an X-ray fluoroscopic imaging apparatus 1 according to Item 1 includes an image display device 11 that displays x-ray images L each showing a predetermined area of interest, and the image display device 11 includes an image display unit 49, an input unit 51, and an image display control unit 57. Information display images D each containing the X-ray image L and image information K are displayed in a row and a column in a matrix on the image display unit 49 of the image display device 11.

The input unit 51 is capable of inputting an instruction to change a position of the X-ray image L and a position of the image information K in the information display image D to display at least two of the X-ray images L adjacently. The image display control unit 57 causes the image display unit 49 to display the X-ray images L adjacently according to the instruction input to the input unit 51.

That is, unlike conventional apparatuses with an unchanged positional relation between the X-ray image L and the image information K in the information display image D, the X-ray fluoroscopic imaging apparatus 1 according to Item 1 is capable of changing the positional relation between the X-ray image L and the image information K in the information display image D. Even when a pattern in which the plurality of information display images D are arranged in the matrix on the image display unit 49 is changed as needed according to a request of the user, a setting to change anew a positional relation between the X-ray image L and the image information K in each of the information display images D can be made such that the X-ray images L are adjacent to each other in the changed arrangement pattern of the information display images D. Accordingly, when the X-ray images L in the plurality of information display images D are visually compared with each other, the arrangement pattern of the information display images D is changed according to the request of the user, and at the same time, presence of the image information K between the X-ray images L to be compared can be reliably avoided. Thus, it is possible to avoid the image information K becoming the obstacle to the motion in visually comparing the X-ray images; therefore, it is possible to relieve the burden caused by the motion in visually comparing the X-ray images.

(Item 2) An X-ray fluoroscopic imaging apparatus according to the present embodiment includes: an X-ray tube that emits an X-ray to a subject; an X-ray detector that detects an X-ray transmitted through the subject; an arm that supports the X-ray tube and the X-ray detector such that the X-ray tube and the X-ray detector are opposite to each other; an arm rotation mechanism that rotates the arm around a predetermined axis; an X-ray image generator that generates an X-ray image using a detection signal output by the X-ray detector; and an X-ray image display device that displays the X-ray image. The X-ray image display device includes: an image display unit that is capable of displaying, in a row and a column in a matrix, information display images each containing an X-ray image display region in which the X-ray image showing a predetermined area of interest is displayed and an image information display region in which information on the X-ray image is displayed; an information display position setting unit that sets a position of the image information display region in the information display image in advance so as to display the X-ray image display regions adjacently according to a pattern with a positional relation in which the information display images are displayed in the row and the column; and an image display control unit that controls the image display unit such that after the pattern with the positional relation in which the information display images are displayed in the row and the column is determined, the position of the image information display region in the information display image corresponds to a position set by the information display position setting unit.

The X-ray fluoroscopic imaging apparatus according to Item 2 includes an image display device 11 that displays X-ray images L each showing the predetermined area of interest, and the image display device 11 includes an image display unit 49, a layout setting unit 55, and an image display control unit 57. Information display images D each containing an X-ray image L and image information K are displayed in the row and the column in the matrix on the image display unit 49 of the image display device 11.

The layout setting unit 55 sets a position of the image information K in the information display image D in advance so as to display the X-ray images L adjacently according to the pattern with a positional relation in which the information display images D are displayed in the row and the column. The image display control unit 57 causes the image display unit 49 to display the X-ray images L adjacently based on the position, set by the layout setting unit 55, of the image information K in the information display image D.

As described above, in the X-ray fluoroscopic imaging apparatus according to Item 2, the position of the image information K in the information display image D can be set in advance before the X-ray image L is generated. That is, since the X-ray images L can be displayed adjacently without a motion in changing the position of the image information K during an ongoing medical operation, it is possible to relieve a burden caused by a motion in visually comparing the X-ray images and also to further reduce a procedure and time required for the medical operation.

(Item 3) In the X-ray fluoroscopic imaging apparatus according to Item 1 or 2, the image display unit displays, in the row and the column, the information display image including a reference image being the X-ray image in which a blood vessel in the predetermined area of interest is visualized, and the information display image including an acquired image being the X-ray image that is generated most recently and in which the blood vessel in the area of interest is not visualized, and the image display control unit causes the image display unit to display the reference image and the acquired image adjacently.

With the X-ray fluoroscopic imaging apparatus according to Item 3, the image display unit 49 displays an information display image DR and an information display image DF in the row and the column. The information display image DR includes a reference image LR, and the information display image DF includes an acquired image LF. The reference image LR is an X-ray image L in which the blood vessel in the predetermined area of interest is visualized. The acquired image LF is an X-ray image L that is generated most recently and in which the blood vessel in the area of interest is not visualized.

The image display control unit 57 causes the image display unit 49 to display the reference image LR and the acquired image LF adjacently. Specifically, the image display control unit 57 changes the position of the image information K in the information display image DR or the position of the image information K in the information display image DF. This enables the user to observe the reference image showing the visualized blood vessel and recognize the position of the blood vessel, and accurately continue the medical operation on the most recent area of interest shown in the acquired image. That is, it is possible to appropriately perform the medical operation by angiographic imaging while avoiding the image information K becoming the obstacle to the motion in visually comparing the acquired image and the reference image.

### Other Embodiments

Examples disclosed herein are illustrative in all respects and are not restrictive. The scope of the present invention encompasses the claims and all modifications within the meaning and scope equivalent to the scope of the claims. By way of example, the present invention can be modified and implemented as follows.
(1) In each of the above-described Examples, the images arranged in the matrix on the image display unit 49 are not limited to the acquired image LF and the reference image LR. As an example, an ultrasound image may be arranged in the row and the column in the matrix together with the acquired image or the reference image. In this case, the user continues the medical operation while visually comparing the acquired image, the reference image, and the ultrasound image.
(2) In the above-described Examples or modified example, as the arrangement pattern to display the plurality of information display images D in the row and the column on the image display unit 49, a pattern in which two information display images D are displayed in the row in the left-right direction G and two information display images D are displayed in the column in the up-down direction H, is exemplified. However, the arrangement pattern is not limited to this 2×2 arrangement pattern. Three or more information display images D may be arranged in the left-right direction G, or three or more information display images D may be arranged in the up-down direction H.
(3) In the above-described Examples or modified example, the configuration of the image information K and the X-ray image L is not limited to being arranged adjacently in the left-right direction G. The position at which the image information K is arranged in the information display image D is the left or the right of the X-ray image L, and this case has been described; however, the configuration is not limited to this. That is, the X-ray image L and the image information K may be arranged in the information display image D adjacently in the up-down direction H.

FIG. 22 illustrates a configuration with a positional relation in which an X-ray image L is arranged below image information K in each of information display images D in an initial state. FIG. 22 also illustrates, as an arrangement pattern for displaying the plurality of information display images D, a pattern in which three information display images D are arranged in a row in the left-right direction G and two information display images D are arranged in a column in the up-down direction. In the initial state illustrated in FIG. 22, as an example, the X-ray images L are adjacent to each other in the left-right direction G in the three information display images D arranged in the upper section, respectively, of the six information display images D. Accordingly, it is possible to avoid the image information K becoming an obstacle to a motion in visually comparing these three X-ray images arranged in the row in the left-right direction G.

However, in the initial state illustrated in FIG. 22, the image information K becomes the obstacle to the motion in visually comparing the X-ray images L in the up-down direction H. That is, the image information K is arranged between the two X-ray images L arranged in the column in the up-down direction, so that the image information K becomes the obstacle when the two X-ray images L are visually compared.

Thus, in a case of the arrangement pattern in the initial state illustrated in FIG. 22, an operation of changing a layout of the three information display images D arranged on the lower section, of the six information display images D, is performed similarly to step S4 according to Example 1 or step F0 according to Example 2. That is, the user operates the input unit 51 or the like, and inputs an instruction to change the positional relation between the X-ray image L and the image information K of each of the three information display images D. Specifically, the user inputs an instruction to change the layout of the information display images D by moving downward the image information K arranged above the X-ray image L to arrange the image information K below the X-ray image L.

The layout setting unit 55 receives the input instruction, and changes the layout setting to, of the six information display images D, arrange the X-ray image L above the image information K in each of the three information display images D arranged in the lower section, while for the layout setting for the three information display images D arranged in the upper section, the initial state is maintained.

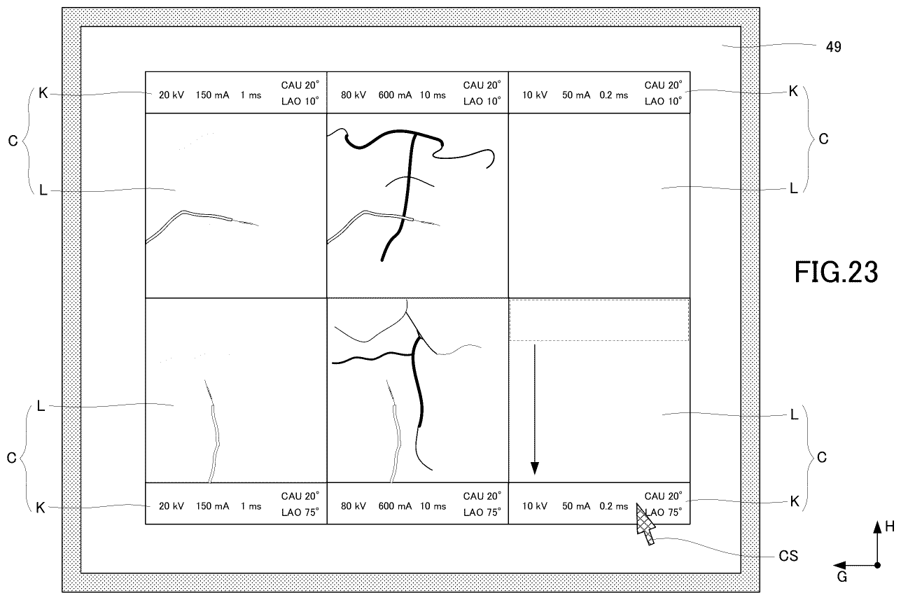

The image display control unit 57 causes the image display unit 49 to display the information display images D in conformity with the layout setting changed through the layout setting unit 55. As a result, as illustrated in FIG. 23, the X-ray image L is arranged below the image information K in each of the three information display images D arranged in the upper section, while the X-ray image L is arranged above the image information K in the three information display images D arranged in the lower section.

Changing the layout setting to the arrangement pattern as illustrated in FIG. 23 allows the X-ray images L to be arranged adjacently also in the up-down direction H. The user can therefore avoid not only the obstacle, caused by the image information K, to the motion in visually comparing the plurality of X-ray images L arranged adjacently in the left-right direction G, but also the obstacle, caused by the image information K, to the motion in visually comparing the plurality of X-ray images L arranged adjacently in the up-down direction H.

### REFERENCE SIGNS LIST

1 X-ray fluoroscopic imaging apparatus
3 table
5 first image pickup mechanism
7 second image pickup mechanism
9 image processing device
11 image display device
13 X-ray tube
15 X-ray detector
16 collimator
17 C-arm
19 slide mechanism
21 rotation mechanism
23 columnar support
25 pedestal
27 X-ray tube
29 X-ray detector
31 C-arm
33 slide mechanism
35 rotation mechanism
39 X-ray emission controller
41 drive controller
43 image generator
45 storage unit
47 console
49 image display unit
51 input unit
53 layout storage unit
55 layout setting unit
57 image display control unit
D information display image
L X-ray image
K image information
LF acquired image
LR reference image

1. An X-ray fluoroscopic imaging apparatus comprising:
an X-ray tube that emits an X-ray to a subject;
an X-ray detector that detects an X-ray transmitted through the subject;
an arm that supports the X-ray tube and the X-ray detector such that the X-ray tube and the X-ray detector are opposite to each other;
an arm rotation mechanism that rotates the arm around a predetermined axis;
an X-ray image generator that generates an X-ray image using a detection signal output by the X-ray detector; and
an X-ray image display device that displays the X-ray image, wherein
the X-ray image display device includes:
an image display unit that is capable of displaying, in a row and a column in a matrix, information display images each containing an X-ray image display region in which the X-ray image showing a predetermined area of interest is displayed and an image information display region in which information on the X-ray image is displayed;
an information display region change instruction unit that inputs an instruction to change a position of the image information display region in each of the information display images displayed in the row and the column on the image display unit, such that at least two of the X-ray image display regions are displayed adjacently; and
an image display control unit that controls the image display unit such that based on a content of the instruction input to the information display region change instruction unit, the position of the image information display region in the information display image is changed to display the X-ray image display regions adjacently.

2. An X-ray fluoroscopic imaging apparatus comprising:
an X-ray tube that emits an X-ray to a subject;
an X-ray detector that detects an X-ray transmitted through the subject;
an arm that supports the X-ray tube and the X-ray detector such that the X-ray tube and the X-ray detector are opposite to each other;
an arm rotation mechanism that rotates the arm around a predetermined axis;
an X-ray image generator that generates an X-ray image using a detection signal output by the X-ray detector; and
an X-ray image display device that displays the X-ray image, wherein
the X-ray image display device includes:
an image display unit that is capable of displaying, in a row and a column in a matrix, information display images each containing an X-ray image display region in which the X-ray image showing a predetermined area of interest is displayed and an image information display region in which information on the X-ray image is displayed;
an information display position setting unit that sets a position of the image information display region in the information display image in advance so as to display the X-ray image display regions adjacently according to a pattern with a positional relation in which the information display images are displayed in the row and the column; and
an image display control unit that controls the image display unit such that after the pattern with the positional relation in which the information display images are displayed in the row and the column is determined, the position of the image information display region in the information display image corresponds to a position set by the information display position setting unit.

3. The X-ray fluoroscopic imaging apparatus according to claim 1, wherein
the image display unit displays, in the row and the column, the information display image including a reference image being the X-ray image in which a blood vessel in the predetermined area of interest is visualized, and the information display image including an acquired image being the X-ray image that is generated most recently and in which the blood vessel in the area of interest is not visualized, and
the image display control unit causes the image display unit to display the reference image and the acquired image adjacently.

4. The X-ray fluoroscopic imaging apparatus according to claim 2, wherein
the image display unit displays, in the row and the column, the information display image including a reference image being the X-ray image in which a blood vessel in the predetermined area of interest is visualized, and the information display image including an acquired image being the X-ray image that is generated most recently and in which the blood vessel in the area of interest is not visualized, and
the image display control unit causes the image display unit to display the reference image and the acquired image adjacently.
